(19) United States
(12) Patent Application Publication
(10) Pub. No.: US 2022/0398735 A1
(43) Pub. Date: Dec. 15, 2022

(54) METHOD AND SYSTEM FOR AUTOMATED PROCESSING, REGISTRATION, SEGMENTATION, ANALYSIS, VALIDATION, AND VISUALIZATION OF STRUCTURED AND UNSTRUCTURED DATA

(71) Applicant: NeuroSimplicity, LLC, Shrewsbury, NJ (US)

(72) Inventors: Jared Rosenblum, Colts Neck, NJ (US); Vikram Chandrashekhar, Trabuco Canyon, CA (US); Vibhu Chandrashekhar, Trabuco Canyon, CA (US)

(73) Assignee: NeuroSimplicity, LLC, Shrewsbury, NJ (US)

(21) Appl. No.: 17/838,311

(22) Filed: Jun. 13, 2022

Related U.S. Application Data
(60) Provisional application No. 63/209,611, filed on Jun. 11, 2021.
(60) Provisional application No. 63/294,916, filed on Dec. 30, 2021.

(51) Int. Cl. G06T 7/00; G16H 30/40; G06T 7/33; G06T 15/08; G06T 19/20; G06T 7/50; A61B 6/00

(52) U.S. Cl. CPC G06T 7/0014; G16H 30/40; G06T 7/344; G06T 15/08; G06T 19/20; G06T 7/50; A61B 6/5235; A61B 6/5258; A61B 6/5217; G06T 2200/08; G06T 2207/10081; G06T 2207/10088; G06T 2207/30004; G06T 2207/20021

(57) ABSTRACT

A method for automated analysis of data obtained from biologic, or non-biologic, material is provided. The method includes extracting, in a visualization of the material, first shapes that combine to form a target shape. The method also includes registering the first shape of the target shape to second shapes of a generic shape, and identifying variations between the first shapes and the second shapes. A system for analyzing biologic material is provided that includes an extraction engine a registration engine an identification engine a display an atlas adapted to provide the generic shape and a database for storing the visualization of the target shape. A non-transitory computer-readable medium storing a program for analyzing biologic material is provided. The program includes instructions that, when executed by a processor, causes a processor to execute the method.

### CROSS-REFERENCE TO RELATED APPLICATIONS

The present application claims priority to U.S. Provisional Patent Application No. 63/209,611, filed Jun. 11, 2021, and U.S. Provisional Patent Application No. 63/294,916, filed Dec. 30, 2021, each of which is incorporated by reference in its entirety.

### BACKGROUND OF THE INVENTION

### 1. Field of the Invention

The invention relates to visualization of a biologic structure of a patient, or a non-biologic structure, and graphic analysis thereof. Imaging of structured or unstructured data can be combined by deformable registration to each other or to a reference atlas, and the input images and or resulting output registered images can be analyzed by shape analysis.

### 2. Description of the Related Art

Data can take many forms (unstructured, for example tabular data, and structured, for example images) and can have any number of dimensions. Dimensions are the number of features associated with a given data element in a dataset (e.g., a three-dimensional RGB image would have 4 dimensions, 3 spatial dimensions, X, Y, and Z, and a 4th dimension for color channel). A data element is a single sample from a dataset with all its associated features (for images, this would be a single pixel/voxel). Unstructured datasets are defined here as datasets where contained data elements do not depend on their relative position to each other (e.g., in tabular data with n rows and d columns, the n rows can be rearranged along that dimension without affecting subsequent analysis). Structured datasets are therefore defined as datasets where contained data elements depend on their relative position to each other (e.g. the intensity of a pixel/voxel in an image depends on the intensities of its neighboring pixels/voxels since an image is a spatial representation of some data). An image is defined herein as any visual depiction of data. Visual depictions of unstructured data can include two-dimensional plots of tabular data.

Unstructured data processing includes but is not limited to conversion of unstructured data to structured data (e.g., two-dimensional plot) or modification of data elements within the dataset (e.g., normalizing the value a dimension/feature such that it has a given range). Structured data processing includes but is not limited to modification of data elements within the dataset (e.g., normalization of pixel/voxel intensity values within an image to be between a given range). Image processing is the modification of an image to achieve a certain goal and is a required step for many visualization and analysis methods.

### SUMMARY OF THE INVENTION

A method for automated analysis of data obtained from biologic, or non-biologic, material is provided. The method includes extracting, in a visualization of the material, first shapes that combine to form a target shape. The method also includes registering the first shape of the target shape to second shapes of a generic shape, and identifying variations between the first shapes and the second shapes.

The registering of the first shapes of the target shape to the second shapes of the generic shape may include identifying marker points in the first shapes that correspond to generic marker points in the second shapes, and aligning the first shapes and the second shapes based on a first optimization function. The registering may also include matching a first contrast of the first shapes with a second contrast of the second shapes by masking at least a portion of at least one of the first contrast and the second contrast. The registering may further include deforming at least one of the first shape and at least one of the second shape based on a second optimization function.

The method may include, prior to the extracting operation, processing input data associated with the visualization by rotating the visualization to a standard orientation, homogenizing an intensity across the image, and/or eliminating artifacts.

The method may include validating the registration by comparing an extracted feature from the visualization to a further extracted feature of a further visualization.

The identifying operation may include identifying local changes within the first shapes and the second shapes, and evaluating the registering using a similarity metric.

The method may include displaying a further visualization of the target shape with data associated with the generic shape as a three dimensional representation. The data associated with the generic shape may be displayed in the further visualization of the target shape in layers selectably displayable by a user. The data associated with the generic shape may include name, function, and connection identifications. The variations between the first shapes and the second shapes may be displayed in the further visualization and may be identified as abnormal based on a model.

The first shapes may include first graphlets, and the first graphlets may include first nodes and first segments. The second shapes may include second graphlets, and the second graphlets may include second nodes and second segments. Alternatively, the first shapes may include first volumetric objects, and the second shapes may include second volumetric objects.

The generic shape may be received from an atlas, and the visualization of the target shape may be obtained by Magnetic Resonance Imaging, Computerized Tomography scan, or a radiologic scan.

The method may include extracting, in a further visualization, third shapes of a further target shape to form a further target shape. The method may also include registering the third shapes of the further target shape to at least one of the first shapes of the target shape and the second shapes of the generic shape.

A system for analyzing biologic material is provided that includes an extraction engine running on a processor coupled to a memory. The extraction engine extracts, from a visualization of the biologic material, or alternatively, non-biologic material, first shapes that combine to form a target shape. The system may also include a registration engine running on the processor. The registration engine registers the first shape of the target shape to second shapes of a generic shape of a generic shape.

The system may further include an identification engine running on the processor. The identification engine identifies variations between the first shapes and the second shapes. The system may include a validation engine adapted to validate the registration output by the registration engine by comparing an extracted feature from the visualization to a further extracted feature of a further visualization.

The system may include a display adapted to display a further visualization of the target shape with data associated with the generic shape as a three dimensional representation. The data associated with the generic shape may be displayed in the further visualization of the target shape in layers selectably displayable by a user. The data associated with the generic shape may include name, function, and connection identifications. The variations between the first shapes and the second shapes may be displayed in the further visualization and may be identified as abnormal based on a model. The system may include an atlas adapted to provide the generic shape and a database for storing the visualization of the target shape.

A non-transitory computer-readable medium storing a program for analyzing biologic, or non-biologic, material is provided. The program includes instructions that, when executed by a processor, causes a processor to execute any of the methods described herein, and/or operate any of the systems described herein.

Understanding physiologic and pathologic organ system function such as in the central nervous system depends on the ability to map entire in situ vasculature and organ interfaces, e.g., cranial vasculature and neurovascular interfaces. To accomplish this, a method and system are provided that combine a non-invasive workflow to visualize murine cranial vasculature via polymer casting of vessels, iterative sample processing and micro-computed tomography with automatic deformable image registration, feature extraction, and visualization. This methodology is applicable to any tissue and allows rapid exploration of normal and altered pathologic states

According to exemplary embodiments of the invention, the following processes may be performed automatically:

process structured or unstructured data of any dimension from multiple modalities/sources to generate visual representations of data or images;

fuse, or integrate multiple images from different (or same) modalities by performing nonlinear (or linear) registration;

extract (or segment) any feature(s) of interest, which may include but is not limited to organ(s) or any other anatomic regions or structures, from images generated from different (or same) modalities;

build or update a compendium of reference object(s) of interest (also referred to herein as an atlas);

analytically and quantitatively compare data (for example, if medical imaging, of the same or different patient and from the same or different timepoints);

analyze and compare extracted object(s) to the atlas, while also updating the atlas in order to automatically detect anatomic malformation and deviation, creating a report for that object; and

visualize (or render) objects extracted from images and their corresponding analytic reports in a shareable, interactive tool.

The order in which is these steps is performed can be modified to suit the information provided and the application. Exemplary embodiments of the present technology extract, analyze, interpret, and visualize information from within the same dataset, data from different sources, acquired at the same time-point or different time-points, from the same or different objects all in the same visualization. This allows for visualization and also, when performing this across longitudinal data or different samples/patients or modalities, exemplary embodiments enable analysis and comparison. This is possible due to the methods and systems described herein for application of automated processing, registration, feature extraction, analysis, validation, and visualization.

Exemplary embodiments of the present technology enable identifying and utilizing features from structured and unstructured data. For example, to obtain a network from vessels from an image of biological material, several things need to be done, including morphological operations and feature extraction using, for example, thresholding-based segmentation methods and deformable registration between multiple images that may include one or more reference atlases. It is important to note that in some instances the order of these steps may vary, as a reference atlas may not be necessary, and different features may be extracted. Vessels can be extracted from an image by simple thresholding if they are the only feature in the image, and that vessel segmentation can then be used to generate the network which is then registered and compared with other networks, including the atlas. However, if vessels are not the only extractable feature in the image, which may be the case in CTs of the head, then not only are more complicated processing steps required, deformable registration to other atlases may be required to pull out those other features first. An atlas may be an available annotated atlas, for example something an expert has created, or may be any other image used for reference in registration. The atlas may be the image to which the first image is deformed or registered.

Exemplary embodiments of the present technology enable handling of multiple types of data through multiple processing steps to enable extraction of certain features. For example, if both vessels and bone are in the image, a bone atlas can be generated from other bone-containing imaging data to register to the image that contains both vessels and bone, to then extract bone. In this way, vessels may be pulled out as a remaining extractable object in the image.

An exemplary method may include the following steps:

1. Input data

2. Cross-modal Deformable Registration of data (invertible and batch)

3. Feature Extraction

4. Building Atlases by combining Registration and Feature Extraction

5. Having each object be separate when extraction allows us to interact with, manipulate or correct individual objects or sub-objects.

Incorporating the above steps in a pipeline (also referred to herein as a workflow pipeline, a workflow, and/or a method) allows for automated analysis and visualization of objects, their sub-objects, and corresponding input data or intermediate outputs. This includes the superimposition of input data, three-dimensional extracted objects, visualization of analysis and reported data, and additional user-specified or process-defined outputs in the same space.

For example, an output of an analysis may be a two-dimensional heatmap of volume change per region of a brain between one image dataset registered to one or more corresponding datasets. The present technology can generate a three-dimensional spatially relevant heatmap that is separated by objects using the same separation criteria as the original object through feature extraction and registration. The present technology also can overlay this output three-dimensional analysis back on the output objects obtained through feature extraction and registration. In this way, The present technology is able to show in an interactive three-dimensional visualization how a sample compares to either other samples or the atlas generated during the batch analysis.

The Cross-modal Deformable Registration of data process may enable a “many” to “many” relationship between the image and object attributes. e.g.:

1. the same object at different timepoints

2. a different object at different timepoints

The Feature Extraction process can be done either on the base data or post-registration data. Once the registration is complete, the calculated transformation may be applied, which identifies how one image differs from another, with respect to the extracted feature or globally across the whole image. This enables further analysis for the extracted feature, either individually or in a batch process.

Shape analysis for extracted objects is also provided by the present technology, analogously to the network based analysis. Further variations include statistical analysis, intensity analysis, and more. The present technology is not limited to MRI, CT, or similar imaging, but can incorporate microscopic data including histology and light-sheet microscopy as well as structured and unstructured data across other domains.

### BRIEF DESCRIPTION OF THE DRAWINGS

The invention is described in more detail with reference to the accompanying drawings, in which only preferred embodiments are shown by way of example. In the drawings:

FIG. 1 is a flowchart illustrating an overview of the exemplary method for automated processing, registration, feature extraction, analysis, validation, visualization, and interaction with structured and unstructured data;

FIG. 2 is a flowchart illustrating a method for determining variation in shapes according to an exemplary embodiment of the present invention;

FIG. 3 is a flowchart illustrating a method according to an exemplary embodiment of the present invention; and

FIG. 4 is a schematic diagram of computing system used in an exemplary embodiment of the present invention.

### DETAILED DESCRIPTION

The present invention pertains to a method and system of handling, processing, registering, analyzing, visualizing, and interacting with structured or unstructured data. The data may be of biologic or non-biologic material. An embodiment of this process includes a method for iterative processing and data acquisition of biologic material and input of this data into the method and/or system.

The exemplary method and system may be used with other sources of data that are not biologic material, such as quality control steps in manufacturing. One such embodiment may include 1) the generation of a physical model from imaging data, 2) imaging this physical model, 3) inputting this imaging data into the system to register it back to the original image to ensure that what has been generated is accurate to the original data. In some alternative exemplary embodiments, patient imaging data may be used, such as for production of a mechanical heart valve or a cranial bone replacement. In still further exemplary embodiments, the system and method described herein may be used in the production of other manufacturing parts. Consequently, an exemplary application of the present technology enables production and quality control for production of any part via imaging of the produced part and comparison with original source data.

Regarding processing of data, image registration may be enabled by a homogenous distribution of intensity in the image such that intensity differences represent true differences. Feature extraction (e.g. segmentation) may be enabled by enhancement or differentiation of the feature(s)/object(s) of interest from other feature(s)/object(s) in the image. Visualization may be determined based on fidelity to the true shape/structure of the object being imaged.

The exemplary system and method may output a shareable interactive report and/or data, which may be used for medical imaging by healthcare providers and/or scientists. The reports and/or data may be used for visualizing and interacting with multiple different modalities of images in the same space and identifying variation in biologic tissues or non-biologic structures. The reports and/or data may also be used for surgical navigation and or robotic surgery for any one of surgical planning, visualization, and/or annotation.

In regard to use of the exemplary methods and systems for manufacturing, the reports and/or data may be used by quality control engineers, artificial intelligence, and/or an autonomous system that does not require a human viewing the output. In this context, the reports and/or data may be used to validate pipeline processes, for quality control and/or assurance, and/or to identify variation in manufactured items.

A non-invasive workflow is provided to visualize in situ vasculature and surrounding anatomy of organ systems such as the murine cranial vasculature, brain, skull, and soft tissues that involves 1) terminal polymer casting of vessels, 2) iterative sample processing and imaging with multiple modalities including micro-CT, and 3) automated deformable cross-modal image registration, feature extraction, and visualization. While developed on cranial vasculature, it can be applied to any image of an organ with contrast such as a polymer-casted organ imaged on micro-CT.

Current methods of visualizing vasculature are limited by 1) poor resolution using in vivo contrasted imaging, 2) invasive or destructive tissue preparation with many ex vivo methods, 3) or lack of relationship of the vasculature to the surrounding anatomy in traditional visualizations of polymer casting. An exemplary embodiment of the workflow, which combines polymer casting, iterative processing and imaging of the same sample, and deformable registration to combine these data, allows visualization of the fine detailed vascular map in the context of the surrounding anatomy in situ.

Exemplary embodiments of the method and system is broadly applicable to many fields. Exemplary embodiments provide a non-invasive visualization of murine cranial vasculature in its entirety with the surrounding anatomy intact, automatically registers cross-modal imaging data, extracts features, and performs analysis. A novel non-invasive approach involving polymer casting of vessels, iterative sample processing and microcomputed tomography (micro-CT), and automatic deformable registration/visualization to construct a high-resolution three-dimensional atlas of murine cranial vasculature in relation to brain, meninges, and surrounding skull bone. The ability to generate detailed and accurate anatomic maps of the entire vascular network that supplies the cranial compartment will greatly advance central nervous system (CNS) research focused on states of health and disease. Even more importantly, this approach can be used to rapidly generate complete vascular maps in any tissue throughout the body.

The methodology allows visualization of all cranial structures and can improve understanding of vascular interfaces that maintain CNS tissue homeostasis as well as alterations that appear during development of neurologic diseases. For example, exemplary methods can be used to automatically detect pathological variation from normal anatomy, identify regions of vascular damage/repair, and detect regions of vascular connectivity that would otherwise be missed with more invasive approaches.

The present technology uses algorithms and software tools to process data, including for example converting unstructured data to structured data. Exemplary embodiments may convert relevant input data, whether structured or unstructured, into an image for subsequent analysis.

Processing for Image Registration

For automated image registration to produce accurate results, the images that are being registered to each other should have some correspondence (for example polynomial intensity) and homogenous spatial distribution of intensity. To accomplish this, a standard intensity inhomogeneity correction method designed for MR imaging can be used. This method can be applied to any imaging modality. Any other processing methods may be used to enhance correspondence between images.

Processing for Feature Extraction

Prior to extracting features of interest, there are processing steps that are typically applied to the image to enhance the separation between the features and/or objects of interest and the remaining image content. For example, to extract blood vessels from an MRI with contrast, a vessel enhancement filter such as the Frangi filter may be used, followed by thresholding to extract the vessels. Current image processing systems and methods require manual intervention and subjective evaluation of the results whereas the exemplary method is fully automated with quantitative evaluation of final outputs without the need for manual intervention. A series of filters may be applied in an automated fashion without the need for manual validation at each step. The present technology combines these image processing steps in a fully automated fashion with the rest of the system and method including feature extraction.

Processing for Visualization

Prior to visualization, it is sometimes required to process imaging data so that the image quality improves. This can be subjective, but in the present system and method quantitative metrics are used that do not require subjective evaluation. One embodiment of a visualization in the present technology may be a full-featured web-based, interactive, and shareable visualization method.

Image Registration

Image registration is the process of computing a transformation that aligns two or more images (for two images, a moving and fixed image, where the moving image is the image being transformed) to each other typically based on manual landmarking of corresponding points and/or pixel/voxel intensity values (landmark free). The produced transformations are typically global (rigid/affine) or local (deformable), via displacement field transformations.

A rigid transformation is a global transformation of an image (affects all pixels/voxels in the image in the same way) that can include translation and rotation (i.e., rigid transformations preserve lengths and angles). An affine transformation is a global transformation of an image that can include translation, rotation, scale, and shear components. A deformable transformation is a local transformation of an image and can produce a different translation, or displacement, for every pixel/voxel in the image. Deformable registration algorithms produce much more accurate transformations than affine or rigid transformations because they can more accurately align local regions of images whereas affine and rigid transformations are global. Deformable registration allows 1) inter-patient registration and 2) longitudinal intra-patient registration and analysis since there are many local changes that may occur in organs of interest over time within the same patient and across patients. Affine and rigid transformations do not allow for this kind of local analysis.

Registration may also be performed by any of the methods described in the paper “CloudReg: automatic terabyte-scale cross-modal brain volume registration”, Vikram Chandrashekhar, et al., Nature Methods, Aug. 1, 2021.

Optimization

Automated image registration requires an image quality metric to determine if a given transformation is optimal. In particular, when image registration is done using pixel/voxel intensity values, an image similarity metric is used to determine the quality of alignment and is iteratively minimized. For intra-modality (within modality) registration, a mean squared error (MSE) metric (squared difference of image intensity) is typically used. For inter-modality (cross modality) registration, a mutual information (MI) error metric is typically used. MI is a metric that relies on the intensity distributions of the two images and therefore operates on histograms of image intensity whereas MSE operates directly on each voxel in the image, producing an error signal at every voxel.

CloudReg, an open-source registration tool under an Apache 2.0 license, uses an image registration cost (or objective) function is created that enables inter-modality registration, like MI, while also producing a per-pixel/voxel error signal, like MSE. This per pixel/voxel error signal is used to enable Inter-modality registration by computing a spatially-varying (per pixel/voxel) polynomial intensity transform from one image to another. This makes possible registration between two images whose intensity distributions can have arbitrary relationships (e.g., corresponding structures in the image can have opposite intensity distributions in local regions of the image). This registration algorithm enables integration of images and information extracted/segmented from them. However, to apply the registration algorithm, the images need to be pre-processed to remove artifacts/artefacts (undesirable alterations to an image due to the physical principles of the technique or damage from sample preparation) unique to each imaging modality with which the images are acquired.

Validation (Manual Landmarking/Segmentation)

Since registration methods cannot be validated using the same image similarity metric used for optimization, validation of registration accuracy is typically computed on the whole registered images either by using corresponding points placed on the images (manual landmarking) or by using corresponding objects segmented from the images (overlap metrics). The transformations computed from the registration method are applied to one set of points (or labeled regions) to bring both (or more) sets of points (or labeled regions) into the same coordinate space. The resulting Euclidean distance between corresponding pairs of points can give an estimate of the registration accuracy in physical units (e.g., millimeters). For corresponding labeled regions, an image overlap metrics is computed including the Intersection over Union (IoU) score, dice coefficient, or F1 score, among others. IoU is a number between 0 and 1 that is the sum of the number of pixels/voxels that are overlapping in both regions divided by the sum of the number of pixels/voxels in both regions. Dice coefficient and F1 scores are scaled versions of the IoU score.

Manual landmarking methods only provide limited assessment of registration accuracy because for deformable transformations, there can be different transformations locally at different locations in the image. Therefore, the manual landmarking method can only sample the true registration accuracy at/around the locations where the landmark points are placed. Manually labeling corresponding regions of the image can address these drawbacks present in landmark accuracy but are significantly more time-intensive and manual processes. These limitations necessitate the development of a novel validation approach.

Registration

A novel automated way to validate image registration in combination with the rest of an exemplary workflow pipeline is provided. The same structures, which can be either from the same sample or different samples, can be imaged using different modalities. Exemplary embodiments segment/extract the same structure from both images from different modalities. The segmented/extracted objects can be transformed to a same space using the transformations computed by a registration algorithm according to the present technology. The objects can be compared to each other using object similarity metrics which include but are not limited to dice coefficient, Intersection over Union (IoU), precision, and recall. Quantitative metrics may be used to assess registration quality as compared with qualitative assessment via visualization.

For example, given an MRI and CT with and without contrast for a single patient, blood vessels from the contrasted images may be segmented and the registration algorithm used to register all the different modalities to each other and display them in the same coordinate space. The segmented blood vessels from the MRI and CT images should match up when transformed to the same coordinate space since they are images of the same object. This built-in correspondence may be used to automatically validate registration accuracy by computing dice coefficient, IoU, or a related overlap metric.

Feature Extraction

Feature extraction, which can include segmentation, from an image is the process of designating a pixel/voxel or group of pixels/voxels that represent a region or volume of interest. This region or volume of interest can be used to create an object (extracted volumetric representation of compiled two-dimensional data). This can be done in a manual or semi-automated fashion.

Manual feature extraction is typically done with a graphical user interface (GUI)-based computer program which allows for delineation of structures from imaging data using a mouse and keyboard connected to a computer. This requires a skilled person to perform the entire process and, for volumetric imaging, requires identifying the region or volume of interest on every two-dimensional slice. This may be an extremely time consuming and subjective process.

While semi-automated feature extraction algorithms, which automate portions of the process, for example, by providing an initial guess of the feature to extract, reduce the amount of manual intervention required, these algorithms are still primarily manual processes. For example, ITK SNAP is a semi-automated segmentation tool for two-dimensional/three-dimensional imaging data that still requires manual intervention at each step.

The present technology builds on the existing methods by combining a variety of processing steps to extract features of interest from imaging data in a fully automated fashion. The exemplary method can generalize well and, in most embodiments, does not require manual intervention as it is a combination of operations applied to the input data.

Build Reference Atlas

A reference atlas for imaging data is an “average” representation of data acquired from images of many different individual objects of the same type. Reference atlases may also contain associated parcellations (or divisions) of the object into sub-objects typically obtained via manual segmentation. Because of these parcellations, reference atlases serve an important role in understanding normal and abnormal morphological (shape-based) variations in objects.

Many reference atlases today are a series of two-dimensional images or reconstructed three-dimensional volumes from serial two-dimensional images parcellated into meaningful regions. Given this, the current process of understanding morphological variations is highly subjective and manual, requiring an expert to compare imaging of a new subject to the atlas on a per slice basis. Newer reference atlases are created with volumetric imaging and parcellated into meaningful regions. For example, there exists a volumetric mouse brain imaging reference atlas with associated brain region parcellations called the Allen Reference Atlas (ARA). There also exists, for example, a volumetric human brain MRI imaging volume that represents many brain images that have been registered to each other, averaged, and parcellated into brain regions.

Newer reference atlases with volumetric imaging and parcellations can be combined with deformable registration to automatically segment newly imaged objects of the same type by region and analyze morphological changes based on the computed transformations.

Exemplary embodiments of the present technology create reference atlases in combination with the rest of the workflow pipeline. To analyze an imaged structure of interest, a reference atlas is needed to determine the deviation of the imaged structure from what is considered “normal”. If a reference atlas does not exist for a given structure, the present technology can aggregate many images, along with corresponding structured or unstructured data, of that structure to create a reference atlas of what is normal for that structure. This reference atlas can be updated with each additional image that is obtained using deformable registration methods described above.

The reference atlas and deformable registration may automatically extract a feature of interest from an image in combination with the rest of the workflow pipeline, particularly the morphological/shape analysis.

If a reference atlas already exists for an object of interest, that reference atlas may be used to perform the subsequent steps below. In one embodiment, a reference atlas of the skull in the human head may be created given CT images of human heads. A threshold-based method may be used, among other filters, to extract the skull from the image and create a mesh representation of the skull. Each skull mesh in the dataset would then be deformably registered to the other skull meshes to produce an average mesh. The volume change at each surface in the mesh can be represented in three-dimensional using a color scale and may contain statistical output including but not limited to mean, and standard deviation information at each face in the mesh. This average mesh may be used as a reference atlas for the human skull.

Alternative exemplary embodiments for extracting features may utilize machine learning to identify abnormalities directly, possibly without using a threshold. Additionally or alternatively, a model may be utilized to extract features, and may include a threshold-based model, a machine learning model, and/or a statistical atlas-based model.

Analysis

There are many ways to analyze extracted features of interest from imaging data and they can vary depending on the application. Typically, this involves taking manual measurements (distances, volumes, etc.) of the extracted features or in the source imaging data.

When combined with image registration, extracted features of interest can be analyzed in an automated and quantitative fashion across the whole image. Using the transformations produced by deformable registrations, local volume changes across an entire object can be determined and categorized by region. Variation between two shapes may be evaluated by computing the local volume change across whole shapes, and this volume change can be compared to a generic shape or otherwise assessed.

Other types of data besides imaging can also be analyzed, for example, graph (or network) objects. A graph is mathematical object that consists of a set of nodes and edges between those nodes. A graph can be used to represent many-to-many relationships like those present in social media networks, neurons/regions in the brain, relationships between species, among many other examples.

Exemplary embodiments: 1) combine deformable image registration with reference atlas creation; and 2) apply shape analysis, which may include connectivity analysis of graph representations, to automate morphological analysis of said objects (determination of normal and abnormal variations).

Shape Analysis

Image registration produces a transformation, or a displacement field, where each pixel/voxel in the image can have a unique displacement (translation vector in X,Y,Z to move that pixel/voxel to its new location in the transformed image). This displacement field can be analyzed by computing its volume change at every pixel/voxel in the image. When combining this with parcellated reference atlases, the total volume change by parcellated region of a reference atlas can be computed after it has been transformed to the input data.

Shape analysis can be defined as morphological analysis of objects in any number of spatial dimensions (e.g. volume change analysis) including networks. One embodiment of shape analysis according to the present technology may have two steps. First, the object is converted, which may include organs or other anatomic regions of interest, into a volumetric mesh (if three-dimensional data). The second step may be a comparison to and update of the reference atlas for the corresponding object. As an example of comparison to a reference atlas, normal and abnormal volume changes can be determined based on typical distributions of volume changes in those regions. To compare objects of the same type, statistical analyses are performed including, but not limited to, comparison of parcellated regions of the objects, specifically to look for regions of difference between the object and the corresponding reference atlas entry. The resultant shape statistical profile is used to update the corresponding atlas and is compared with the atlas to provide automated anatomical malformation and deviation detection.

Another embodiment of shape analysis is connectivity analysis. Connectivity analysis according to the present technology may have three central steps. First, the object of interest, for example vasculature, is converted into a skeletonized form. A skeleton of an object is a 1 pixel/voxel-wide representation of an object (e.g., a stick figure representation of the human body). During, the process of skeletonization, object attributes, including the thickness/radius information, are preserved and included as node or edge attributes in the following step. Second, the skeleton is converted into a graph. In order to generate a graph, at least two pieces of information are needed: the list of nodes, and the list of edges (connections between nodes). The list of nodes may be generated from the voxels comprising the vascular segmentation, and the list of edges may be generated from a thresholding-based nearest neighbor method or any similar edge-finding method. This graph may be further simplified by only representing bifurcation points, for example, as nodes. The last step is comparison to and update of the reference atlas (for example, a reference atlas for vascular networks). To compare vascular networks, graphlets, which are small repeatable subgraph units, are counted. A subgraph is a set of nodes and associated edges for a subset of the graph. Each graphlet represents a unique connectivity configuration. For example, this could be a small vascular region. Graphlet counts and ratios of different graphlets, which may or may not have the same number of nodes and edges, are calculated across the entire vascular graph in order to generate a vascular graph profile for that individual sample. This graph profile is then used to update the atlas and is compared with the atlas in order to provide automated vascular malformation and deviation detection.

The exemplary embodiment discussed above related to vascular connectivity analysis, however the methodology can apply to any type of shape or connectivity data including axonal pathways in the brain (e.g. from diffusion tensor imaging (DTI)).

Visualization

Visualization of data is displaying it or any processed version of it so that it can be viewed. There are existing methods for visualization. These existing methods present challenges including difficulty displaying and interacting with very large data. There are existing methods that perform some subset of the above steps including processing, registration, segmentation, analysis, and visualization but they still require manual intervention and lack generalizability to other types of data (objects/modalities/organ systems) [miracl, 2,3,4,5,]. The next step in the exemplary workflow pipeline involves visualization of all the following in the same coordinate space: registered multi-modal imaging data, their associated segmentation(s), and their respective object profiles in an interactive, web-based visualization. The present technology combines this feature with the rest of the workflow pipeline and fully automates the process end-to-end.

In the case of medical images and segmenting blood vessels, for example, the visualization will contain a three-dimensional display of all registered images and associated object(s) of interest including the network and abnormalities.

### Example—Medical Image Evaluation

Exemplary embodiments of the present technology provide a system and method for evaluating a medical image. Given four volumetric medical images of the same patient without significant time (for example, less than 3 months) in between scans. These may be MR and CT images with and without contrast within the blood vessels. These four acquired images are the inputs to the following steps in this embodiment of the exemplary system and method. The following steps do not necessarily need to happen in sequential order, some steps can be performed in parallel or out of order.

1.1 Pre-Processing

Both the non-contrasted and contrasted MR images may be intensity corrected prior to registration, segmentation, and visualization. The contrasted MR image will be additionally processed by the Frangi filter to highlight the contrasted vessels.

Both the non-contrasted and contrasted CT images will be intensity corrected prior to registration, segmentation, and visualization. CT images will also be processed to remove CT-specific artifacts including but not limited to ring, windmill, and beam hardening. The contrasted CT scan is processed to enhance vessels using a combination of filters including but not limited to the Frangi filter.

1.2 Registration

The intensity corrected MR image without contrast is registered to a human brain atlas (MNI atlas for example) using deformable image registration methods. The intensity corrected MR image without contrast is also rigidly registered to the intensity corrected MR image with contrast. The intensity corrected MR image with contrast is registered to the CT image with contrast. The CT image without contrast is registered to an existing human skull atlas (or one is created using an average of many samples). The CT image without contrast is rigidly registered to the CT image with contrast.

1.3 Segmentation

A final visualization according to the exemplary method and system will contain, but is not limited to, renders of the brain (segmented by region), bone (segmented by region), and vessels of the head (segmented by name and type). Segmentation of the brain by region is enabled by the exemplary deformable registration of the MR image without contrast to a parcellated reference atlas. The registration produces a labeled parcellation of the input MR image without contrast. Segmentation of the bone is done via registration to a skull atlas (if one exists) or from the CT image without contrast directly and is performed using denoising, thresholding, and morphological operations. Segmentation of the vessels of the head is performed using thresholding and morphological operations applied to the pre-processed MR and CT images with contrast.

Validation of the vessel (or brain/bone) segmentation algorithm is performed by manually/semi-automatically segmenting the structure of interest across a predetermined number of samples to obtain its accuracy.

1.4 Build Reference Atlas

Exemplary embodiments of the present technology use the segmentation algorithm discussed above to segment vessels, skull, brain, and other structures of interest from the appropriate input image. A registration algorithm to register each component to a corresponding reference atlas or create one and iteratively update it with each new set of patient MR and CT scans. There exist reference atlases for brain. Reference atlases with associated volumetric imaging data for the vessels of the head and the skull may not be available. Exemplary embodiments of the present technology may be used to create these atlases using large groups of imaging data and parcellating them into meaningful regions/vessels/bone.

As each vessel segmentation from a new patient is obtained, it is deformably registered to the current reference atlas of the vessels which is updated based on the variation present in the new vessels.

1.5 Analysis

Exemplary embodiments of the present technology apply skeletonization and graph generation to the segmentation of the vessels of the head. By combining this with registration and reference atlas information, the present technology can automatically detect vascular malformations, compute volume differences across the whole brain and by specific regions, and compute volume differences across the whole skull and by specific regions (among other possible analyses). The analyses are used to create a report of malformations and other anatomical abnormalities and is prepared for visualization.

1.6 Visualization

Visualization is performed using a web-based, interactive GUI, like Neuroglancer, but with added functionality including real-time computed layers for rendering and real-time pixel-perfect annotations with a custom state-saving function to enable link shortening and sharing of views to medical imaging data in a HIPAA-compliant fashion.

The Figures are described in detail as follows.

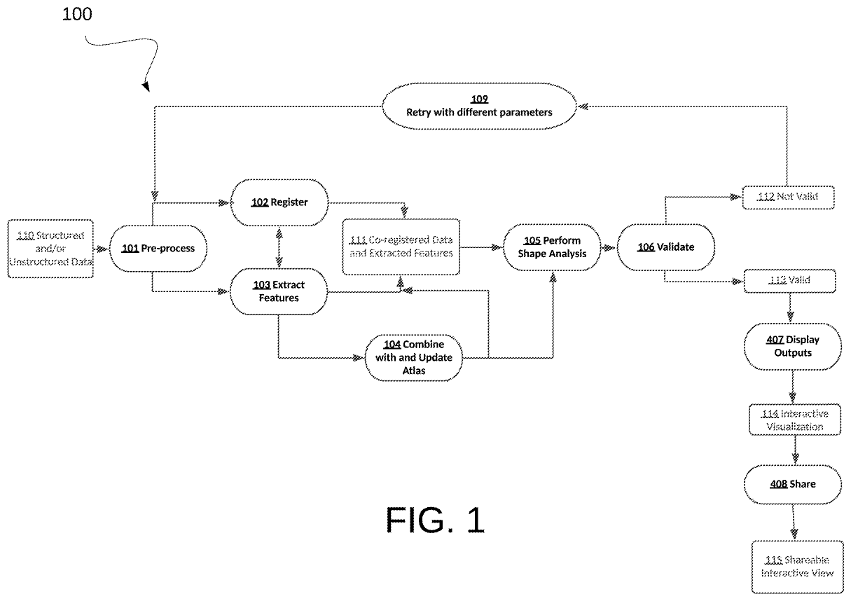

FIG. 1 is a flowchart illustrating an overview of method 100 for automated processing, registration, feature extraction, analysis, validation, visualization, and interaction with structured and unstructured data. The flow in method 100 begins at operation 110 which is an input of structured and/or unstructured data. From operation 100, the flow proceeds to operation 101, which is a pre-processing step. Operation 101 includes performing an initial processing on the structured and/or unstructured data to enable subsequent processing/analysis. From operation 101. Operation 102 is a registering operation, which may include transforming all the input data to the same space by using extracted features, and/or may include a transformation to extract features from the input data. Operation 102 may further include aligning two or more data elements and/or objects of any dimension with one another, to identify a correspondence. Operation 103 may include identifying and highlighting features of interest within the input data using the transformations from operation 102, and/or may include extracting features of interest directly from the input data, for instance by identifying and isolating data and/or objects.

The flow of method 100 may proceed from operation 102 to operation 103, or from operation 102 to operation 111, which indicates to co-register data and extracted features. The flow from operation 103 may proceed from operation 102 to operation 103, from operation 103 to operation 111, or from operation 103 to operation 104, which indicates to combine the processed data with an atlas, or to update the atlas using the processed data. Operation 104 may further include creating or updating a consensus object for each feature and/or object extracted from the input data. From operation 104, the flow in method 100 proceeds to operation 111 and/or to operation 105, which indicates to perform shape analysis. The shape analysis of operation 105 may include determining a variation in extracted objects relative to a consensus object, for example from an atlas.

Operation 111 outputs data to operation 105. From operation 105, the flow proceeds to operation 106, which indicates a validation step. The validation step ensures that the previous processes produce meaningful, accurate results, and may include evaluating any image similarity metric, for example those discussed above. In method 100, operation 106 outputs to operation 112 when operation 106 indicates the analysis is not valid, and operation 106 outputs to operation 113 when operation 106 indicates the analysis is valid. From operation 112, the flow then proceeds to operation 109, which indicates to change the parameters and repeat the method. The flow from operation 109 proceeds to operation 102. From operation 113, the flow then proceeds to operation 407, which indicates to display the data. The displaying operation may include displaying input data, extracted features and/or objects, and/or analysis in the same space, for example in a single image include selectable and variably visible layers. From operation 407, the flow proceeds to operation 114, which indicates to provide an interactive visualization for the user. From operation 114, method 100 proceeds to operation 408, which enables the user to share the visualization and/or the data. Operation 408 the flows to operation 115, which provides a shareable interactive view.

Data is modified, processed, and transformed in exemplary methods. For example, microCT images that are input may be reoriented by rotating the image(s) to a standard orientation. Other examples of modifying/processing/transforming include homogenizing the intensity across the image, eliminating artifacts, etc.

Validation of registration and the other pipeline steps using the same extracted feature that is present in multiple separate input scans. This is the same feature or multiple different features (e.g. vasculature or vasculature and bone from the same person) imaged with multiple different modalities (e.g. MRI, CT, PET, Ultrasound, etc). For example, four different scans may be uploaded in which some scans contain brain only, some contain bone, vessels, and brain, some contain bone and vessels, and some contain vessels. Exemplary embodiments validate the end result by transforming all scans to the same space and then comparing the overlap between bone in the scans that contain bone, brain from the scans that contain brain, and vessels from the scans that contain vessels. These comparisons would provide validation for all the steps of the exemplary process.

This process is explained by the following example. With two images in which the first image has a line and a box and the second image has a line only, but the same line as is in the first image. The exemplary method may extract the box and line from the first image, and extract the line from the second image. In addition, if there is overlap between the box and the line in the first image, the registration to the second image containing the line can be used to determine the location of the box in the first image. The first image may be registered to the second image by putting both the first image and the second image in the same space. To validate, the exemplary method compares the line from the second image to the line from the first image when they are both in the same space. A perfect registration would mean perfect overlap of the line between the first image and the second image. Any deviation from this would indicate misalignment. This misalignment may be compared by a threshold deviation value, form a basis for a deformation, or may be used to evaluate the registration.

Another example also used a first image and a second image, in which the first image has a line, box, and circle, and the second image has the same line and the same box. Exemplary methods may extract the box, line, and circle from the first image, and may extract the line and box from the second image. Similar to the example above, if there is overlap between the circle and either the line or box, the registration of the second image to the first image can be used to determine the location of the circle in the first image. Exemplary methods may register the first image to the second image by putting both the first image and the second image in the same space. In this case, to validate, the line and the box from the second image are compared to the line and the box from the first image. In this case, a perfect registration would mean perfect overlap of the line and the box between the first image and the second image, and any deviation from this would indicate misalignment.

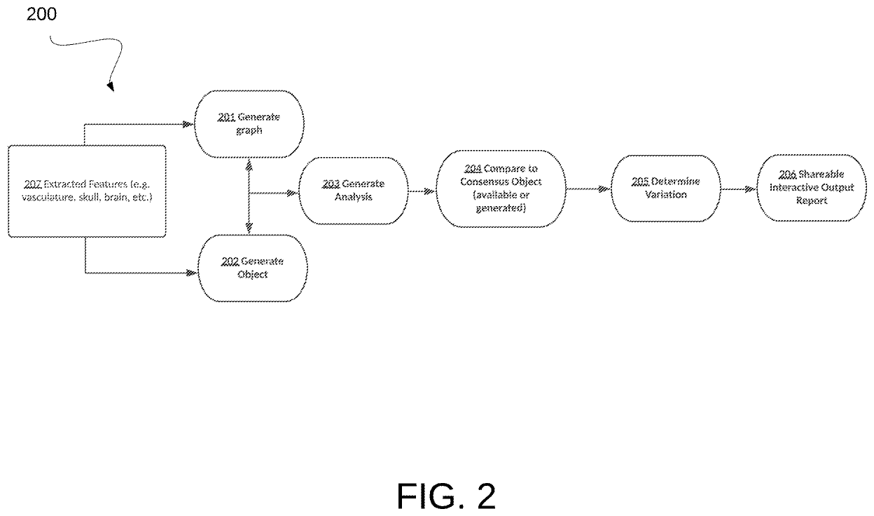

FIG. 2 is a flowchart illustrating method 200 for determining variation in shapes according to an exemplary embodiment of the present invention. The flow in method 200 begins with operation 207, which includes receiving input data related to extracted features, for example vasculature, a skull, a brain, etc. From operation 207, the flow proceeds in parallel to operations 201 and 202. Operation 201 indicates to generate a graph, and includes converting the extracted feature and/or generated object to a graph representation. Operation 202 indicates to generate an object, and includes converting the extracted feature and/or generated graph to an object. Operations 201 and 202 may bilaterally exchange data, and both may output to operation 203, which generates an analysis, including performing shape analyses on the generated objects and/or graphs. The analysis may involve creating structured and unstructured outputs from a shape (defined herein as including at least objects and graphs) to characterize the shape and enable comparison with other shapes. From operation 203, the flow proceeds to operation 204, which indicates to compare the shape-analyzed data to a consensus object (available or generated). Operation 204 may include performing shape comparisons between generated objects and/or graphs and consensus objects and/or graphs. In method 200, the flow proceeds from operation 204 to operation 205. Operation 205 is a determination of the variation in objects based on the previously performed comparison. From operation 205, the flow proceeds to operation 206, which is a shareable interactive output report. The output report may summarize information from some or all of the previous processes.

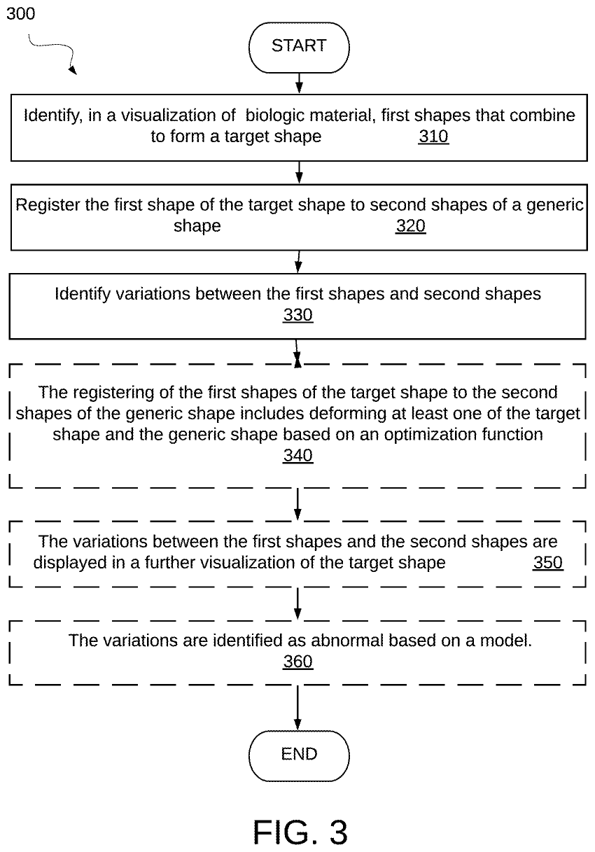

FIG. 3 is a flow chart illustrating method 300 according to the present invention. In FIG. 3, optional steps in method 300 are shown in dotted boxes. The flow in method 300 flows from the start oval to operation 310, which indicates to identify, in a visualization of biologic material, first shapes that combine to form a target shape. From operation 310, the flow in method 300 proceeds to operation 320, which indicates to register the first shape of the target shape to second shapes of a generic shape. From operation 320, the flow in method 300 proceeds to operation 330, which indicates to identify variations between the first shapes and second shapes. From operation 330, the flow in method 300 proceeds to optional operation 340, which indicates that the registering of the first shapes of the target shape to the second shapes of the generic shape includes deforming at least one of the target shape and the generic shape based on an optimization function. The optimization function may be performed on a pixel by pixel (or voxel by voxel) basis. From optional operation 340, the flow in method 300 proceeds to optional operation 350, which indicates that the variations between the first shapes and the second shapes are displayed in a further visualization of the target shape. From optional operation 350, the flow in method 300 proceeds to optional operation 360, which indicates that the variations are identified as abnormal based on a model. From optional operation 360, the flow in method 300 proceeds to the end oval.

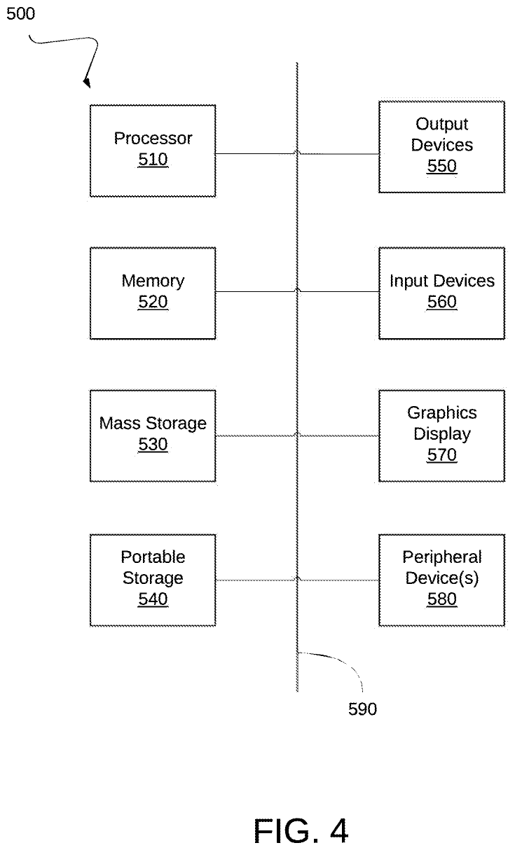

FIG. 4 is a schematic diagram of computing system used in an exemplary embodiment of the present invention. FIG. 4 illustrates exemplary computing system 500, hereinafter system 500, that may be used to implement embodiments of the present invention. The system 500 may be implemented in the contexts of the likes of computing systems, networks, servers, or combinations thereof. The system 500 may include one or more processors 510 and memory 520. Memory 520 stores, in part, instructions and data for execution by processor 510. Memory 520 may store the executable code when in operation. The system 500 may further includes a mass storage device 530, portable storage device(s) 540, output devices 550, user input devices 560, a graphics display 570, and peripheral device(s) 580.

The components shown in FIG. 4 are depicted as being connected via a single bus 590. The components may be connected through one or more data transport means. Processor 510 and memory 520 may be connected via a local microprocessor bus, and the mass storage device 530, peripheral device(s) 580, portable storage device 540, and graphics display 570 may be connected via one or more input/output (I/O) buses.

Mass storage device 530, which may be implemented with a magnetic disk drive or an optical disk drive, is a non-volatile storage device for storing data and instructions for use by processor 510. Mass storage device 530 may store the system software for implementing embodiments of the present invention for purposes of loading that software into memory 520.

Portable storage device 540 operates in conjunction with a portable non-volatile storage medium, such as a floppy disk, compact disk, digital video disc, or USB storage device, to input and output data and code to and from the system. The system software for implementing embodiments of the present invention may be stored on such a portable medium and input to the system 500 via the portable storage device 540.

User input devices 560 provide a portion of a user interface. User input devices 560 may include one or more microphones, an alphanumeric keypad, such as a keyboard, for inputting alpha-numeric and other information, or a pointing device, such as a mouse, a trackball, stylus, or cursor direction keys. User input devices 560 may also include a touchscreen. Additionally, the system 500 as shown in FIG. 4 includes output devices 550. Suitable output devices include speakers, printers, network interfaces, and monitors.

Graphics display 570 may include a liquid crystal display (LCD) or other suitable display device. Graphics display 570 receives textual and graphical information, and processes the information for output to the display device.

Peripheral devices 580 may be included and may include any type of computer support device to add additional functionality to the computer system.

The components provided in the system 500 are those typically found in computer systems that may be suitable for use with embodiments of the present invention and are intended to represent a broad category of such computer components that are well known in the art. Thus, the system 500 may be a personal computer, hand held computing system, telephone, mobile computing system, workstation, server, minicomputer, mainframe computer, or any other computing system. The computer may also include different bus configurations, networked platforms, multi-processor platforms, etc. Various operating systems may be used including Unix, Linux, Windows, Mac OS, Palm OS, Android, iOS (known as iPhone OS before June 2010), QNX, and other suitable operating systems.

It is noteworthy that any hardware platform suitable for performing the processing described herein is suitable for use with the embodiments provided herein. Computer-readable storage media refer to any medium or media that participate in providing instructions to a central processing unit (CPU), a processor, a microcontroller, or the like. Such media may take forms including, but not limited to, non-volatile and volatile media such as optical or magnetic disks and dynamic memory, respectively. Common forms of computer-readable storage media include a floppy disk, a flexible disk, a hard disk, magnetic tape, any other magnetic storage medium, a CD-ROM disk, digital video disk (DVD), Blu-ray Disc (BD), any other optical storage medium, RAM, PROM, EPROM, EEPROM, FLASH memory, and/or any other memory chip, module, or cartridge.

The present technology further enables understanding physiologic and pathologic central nervous system function by mapping in situ cranial vasculature and neurovascular interfaces. Exemplary embodiments provide a non-invasive workflow to visualize murine cranial vasculature via polymer casting of vessels, iterative sample processing and micro-computed tomography, and automatic deformable image registration, feature extraction, and visualization. This methodology is applicable to any tissue and allows rapid exploration of normal and altered pathologic states.

The entire intact murine cranial vasculature has not yet been visualized. Understanding normal cerebrovascular anatomic relationships is critical to the study of intracranial pathologies. Current in vivo contrast-based imaging methods for mice, such as micro-computed tomography (Micro-CT) or magnetic resonance imaging (MRI), are limited in resolution of fine vasculature due to motion artifact and inadequate contrast filling. Optical sectioning using light-sheet microscopy, which is a high-resolution ex-vivo alternative for imaging the brain, can resolve fine cerebrovasculature but cannot presently be performed on the whole head with the skull intact while preserving the sample for further investigation. A workflow is provided to non-invasively and non-destructively generate high-resolution maps of the murine whole-head vasculature and the surrounding anatomy using terminal vascular polymer casting, iterative sample processing, and high-resolution ex-vivo Micro-CT.

### INTRODUCTION

An understanding of central nervous system function during states of health and disease depends critically on the ability to generate detailed and accurate anatomic maps of the entire vascular network that supplies this compartment. The translational study of cranial murine disease models requires a standardized visualization method that contextualizes the entire vasculature in situ relative to the surrounding anatomy, and would greatly advance understanding of neurovascular interfaces. In-vivo contrast-based angiography is standard for defining these relationships in living larger animals and humans. However, due to low resolution and artifacts of in-vivo image acquisition in mice, it is difficult to image and visualize cranial vasculature as it interfaces with related functional tissues, such as the brain, meninges, and skull.

As an alternative to in-vivo contrast-based angiography, casting of vessels with radio-dense polymers has traditionally been combined with tissue or organ dissection, digestion, and dye immersion, along with ex vivo image acquisition. Typically, high-density, radio-opaque polymer mixtures used in these methods are not optimized for arterio-venous transit, which limits their use to casting of either the arterial or venous system. Conversely, low-density radio-opaque polymers that cross capillary beds are not well visualized in imaging methods such as micro-computed tomography (Micro-CT) without prior isolation of the tissue and clearing or digestion, which can distort and/or destroy the gross or fine anatomy of the vasculature. In addition, these methods are limited by destruction of the sample tissue, limited perfusion, or inadequate visualization. While newer tissue clearing and immunostaining immersion-based visualization techniques, such as Clear Lipid-exchanged Anatomically Rigid Imaging/immunostaining-compatible Tissue hYdrogel (CLARITY) and light sheet microscopy, provide very high resolution, three-dimensional (3D), intact images, these methods may produce artifacts such as tissue deformation and illumination inhomogeneity. Further, these techniques still require dissection and removal of the brain, which can distort the anatomy and precludes the study of the entire cranial vasculature. While advances in tissue clearing and light-sheet microscopy have made it possible to image vasculature within bone or to survey large intact regions of mice for specific anatomic structures of interest, the processing in these methods requires specific considerations for downstream investigations such as histology, immunohistochemistry, and molecular genetic techniques, among others.

To visualize the entire murine in situ cranial vasculature in relation to the surrounding anatomy and preserve the sample for further investigation, a non-invasive, non-destructive visualization method combining 1) low-density polymer casting with arterio-venous transit, 2) iterative sample processing and Micro-CT, and 3) automatic deformable registration and three-dimensional visualization through the Neurosimplicity Imaging Suite is provided. The workflow enables non-invasive construction of a high-resolution three-dimensional map of murine cranial vasculature in relation to the brain, surrounding skull bone, and soft tissues.

Overview of Workflow for Visualization of Cranial Vasculature

To ensure capillary transit, an anticoagulant, heparin, is injected and the mouse is allowed to ambulate before atraumatic sacrifice. The descending aorta is then exposed and catheterized, and the inferior vena cava (IVC) is sectioned and sodium nitroprusside is perfused retrograde through the aorta until it exits the IVC. This step clears blood from and maximally dilated all vessels. Next, a low-density, radio-opaque polymer is perfused through the same catheter until it exits the IVC. Finally, the IVC is ligated and the skull exposed to visualize the diploic veins, and perfusion is continued until the diploic veins are visibly filled. This serves as the endpoint for intracranial filling of vessels.

Following curing and fixation, the sample is processed and imaged via Micro-CT at three stages to specifically capture the vascular cast, bone, and soft tissues. An initial Micro-CT is acquired before decalcification that shows bone and the vascular cast. The sample is decalcified to make bone radiolucent and then a second Micro-CT is acquired. This reveals diploic and emissary vessels within the bone and increases visibility of intracranial vessels. Finally, the sample is immersed in phosphotungstic acid (PTA), which binds protein in a concentration dependent manner and makes all tissues visible on a third acquired Micro-CT. The acquired Micro-CT data and the appropriate atlas (for example, the Allen Reference Atlas Common Coordinate Framework Version 3 (ARA CCFv3)) are then deformably registered to the same coordinate space. Features of interest including annotated brain regions, bone, and vessels are automatically extracted and visualized in three dimensions using an imaging application.

Iterative Processing Steps are Validated by Micro-CT

Multiple Micro-CT image datasets are acquired on the same sample following iterative processing to allow the visualization of specific anatomic features including vessels, bone, brain, and other soft tissues. Micro-CT is utilized following each step to determine successful processing, such as complete decalcification and diffusion of PTA. Micro-CT images are used to determine successful iterative processing.

Acquired Micro-CT is Registered and Visualized Together

With these three datasets, the first Micro-CT image is deformably registered with bone and vessel to the image of the decalcified sample. The Micro-CT image is also registered following PTA immersion to the image of the decalcified sample. Finally, all three of these scans are registered and displayed in the same space. This enables visualization of the segmented vessels, brain regions, and surrounding anatomy in three-dimensional.

### Discussion

To evenly cast all the vessels in the head, a method of systemic low-density polymer perfusion is provided. Low-density Microfil® may have advantages over other contrast agents in perfusion of both the intracranial arterial and venous vasculature. The method may be modified in three ways: 1) a lower density polymer mixture is used to ensure non-destructive capillary transit, 2) perfusion is retrograde through the descending aorta to ensure even filling of the anterior and posterior cranial circulation, and 3) a closed system is created to allow for backfilling of the venous vasculature of the entire head. While other polymers can be optimized for arterio-venous transit, Microfil® polymer may be used because others such as vinylite have unfavorable polymerization and curing properties including expansion and heat release that may damage fine vasculature. The exemplary perfusion method may be combined with Micro-CT showed the major cranial vessels in relation to the skull.

An iterative sample processing and Micro-CT approach to visualize the vessels within bone and neurovascular interfaces is provided. Following a first round of Micro-CT to visualize cranial bone and polymer-casted vessels, the same sample is decalcified and Micro-CT is repeated to generate an image of the isolated cranial vasculature. From this decalcified scan, a segmentation is rendered of the in situ cranial vasculature separate from bone. Next, the same sample is immersed in PTA and a third round of Micro-CT is performed to generate an image containing bone, vessels, and soft tissue. Exemplary methods then register, deformably and automatically, all three scans and the Allen Reference Atlas brain region annotations into the same space. Features of interest may be extracted for visualization of the brain, bone, and vessels using an imaging application according to the present technology.

Using only the first round of the workflow described above, abnormalities in cranial vasculature and surrounding bone in a mouse model of Pacak-Zhuang syndrome may be characterized. Through clinical investigations of patients with this syndrome, the vascular malformations in these patients may be recognized as primarily venous and involving both vessels of the brain and the rest of the head. However, further sample processing may be required to visualize neurovascular interfaces with casting and Micro-CT alone. Thus, the present technology provides a non-invasive, non-destructive iterative sample processing and Micro-CT workflow that allow visualization of vessels, soft tissue, and bone separately. The acquired data can be converted to Hounsfield units, a commonly used linear rescaling, if samples of water and air are also acquired with the same parameters on the same machine. Quantitative analysis and measurements, however, can be performed on the acquired data, which is measured in attenuation, because the relative densities of regions within the sample are still the same. An example of quantification that can be performed using the exemplary method includes measuring the length and diameters of the basilar arteries in wild-type mice and the mouse model of EPAS1-Gain-of-Function syndrome. This examination reveals that the mutant B2 and B3 segments of the basilar artery are significantly smaller than the wild-type.

The exemplary methods of the present technology, when combined with registration and three dimensional visualization, offers an unprecedented understanding of the anatomy, particularly neurovascular interfaces. Conventional imaging of vasculature in bone and evaluation of structures of interest over large intact regions of mice is possible in tissue clearing and light-sheet microscopy, these methods are still limited in visualizing all of the structures within the entire intact head, e.g., brain parenchyma, bone, and vessels. Further, while these methods are destructive and preclude further downstream investigation using standard methods such as histology, immunohistochemistry, and molecular genetic techniques, the present technology preserves the sample, enabling further investigations. Since the vasculature is not perfused with fixative in the casting method according to the present technology, fixation parameters can be chosen and optimized for additional tissue studies. The high-resolution in situ visualization afforded by this non-invasive, non-destructive approach should also aid future studies focused on analyzing regions of interest, such as specific neurovascular interfaces.

Maps of cerebrovsaculature at high resolution have been obtained by combining optical methods such as light sheet microscopy with tissue clearing methods. However, prior art methods used may require isolation of the brain from the bone and surrounding tissues. Recent advances in this methodology may allow visualization of vasculature within bone or evaluation of anatomy of interest over large regions of mice using light-sheet microscopy. However, these techniques still have unique sample preparation considerations for optimizing visualization of multiple markers of interest using antibody-based labelling and allowing for further downstream use of the sample. Exemplary methods of the present technology, which is non-invasive and non-destructive, utilize polymer to cast and define vessels and phosphotungstic acid to bind protein in all tissues in a concentration-dependent manner. These two nonspecific methods of labelling tissues therefore allow visualization of all structures within the head without removal of the brain. This method therefore allows study of the entire, intact cranial vasculature. In addition, by iteratively processing and imaging the same sample, the exemplary method can visualize the interfaces of vasculature with regions of tissues of interest in an unprecedented manner.

Using an exemplary tool for automated deformable image registration, feature extraction, and visualization, iteratively processed samples can be combined such that brain, bone, and vessels from the same sample can all be visualized in the same coordinate space. The present technology can handle raw data files including bitmap, TIF, and DICOM. Further, the tool can be used to automatically register the images from a sample to the anatomic parcellations of the ARA CCFv3, allowing brain-region-level annotation. Additionally or alternatively, other reference atlases can be used. Micro-CT enables intact, non-invasive, non-destructive visualization of the whole sample, and also higher resolution.

In conclusion, the present technology provides anon-invasive, non-destructive approach for visualizing the in situ murine cranial vasculature in its entirety with surrounding anatomy intact. The exemplary method improves upon shortcomings of past vascular casting and visualization methods by combining even casting of the entire cranial vasculature, iterative sample processing and Micro-CT, and automatic deformable registration, feature extraction, and visualization. This method enables development of 1) a murine cranial vascular reference atlas, 2) analytical parameters derived from this atlas, and 3) objective methods to standardize the evaluation of cranial vascular disease in murine models. The use of the present exemplary method, which can be applied to any tissue, allows for the rapid exploration and further understanding of normal and disease states.

The exemplary method provided herein is particularly suited for morphological, structural, and developmental studies of vasculature and surrounding anatomy. Using the exemplary vascular casting method, vascular malformations have been identified in a mouse model of Pacak-Zhuang syndrome that recapitulated findings in the in vivo human studies. Further, the exemplary method is not restricted to cranial tissue and can be applied to any organ or tissue of interest to perform similar analyses.

The present technology may also be used with unstructured data. In order to practice the method on unstructured data, unstructured data is first converted to structured data and then the remaining pipeline steps (including deformable registration) are performed on the structured data.

Conversion of unstructured data to structured data can occur in several ways. One way is conversion of tabular data to some two-dimensional or three-dimensional plot/graph/visualization. This visualization can then be used to perform some or all of the subsequent steps of the pipeline. Alternatively, structure can be imposed on unstructured data by sorting all rows by the value in a single column. This allows the sorted result to be processed directly since deformable registration and the other steps of the pipeline can be performed on data of any dimension; that is, even one-dimensional data (including tabular data) can be registered to other datasets of any dimension including one-dimensional data.

A first example starts with two input unstructured datasets. Unstructured Dataset 1 contains two columns with numerical values for Features 1 and 2 for Sample 1. Unstructured Dataset 2 also contains two columns with numerical values for Features 1 and 2 for Sample 2. The exemplary method converts both of these datasets into structured data in the form of a two-dimensional plot. Processing for these datasets can involve removing outliers, normalizing values to their mean, among others. Feature extraction for these datasets can involve computing descriptive statistics including but not limited to mean, median, mode, variance, and interquartile ranges for Features 1 and 2. Registration for these datasets can involve deformably aligning the two-dimensional visualizations to each other. Analysis for these datasets can involve statistical comparison of descriptive statistics between Unstructured Dataset 1 and Unstructured Dataset 2 and identifying variations between the two-dimensional structured representations of these datasets. Interactive visualization for these datasets can involve displaying the two-dimensional structured representations of the input unstructured datasets in the same space and in their native space.

A second example also starts with two input unstructured datasets. Unstructured Dataset 1 contains 2 columns with numerical values for Features 1 and 2 for Sample 1. Unstructured Dataset 2 also contains 2 columns with numerical values for Features 1 and 2 for Sample 2. Structure is imposed on the unstructured datasets by sorting all rows by the values for Feature 1. The exemplary methods discussed above regarding structured data may then be applied to this structured tabular data. Similar to the example above, processing for these datasets can involve removing outliers, normalizing values to their mean, among others. Similar to the example above, feature extraction for these datasets can involve computing descriptive statistics including but not limited to mean, median, mode, variance, and interquartile ranges for Features 1 and 2. For these datasets, registration can involve deformably aligning each corresponding one-dimensional dataset from Unstructured Datasets 1 and 2. Feature 1 from Unstructured Dataset 1 is deformably aligned to Feature 1 from Unstructured Dataset 2 and Feature 2 from Unstructured Dataset 1 is deformably aligned to Feature 2 from Unstructured Dataset 2. Analysis for these datasets can involve statistical comparison of descriptive statistics between Unstructured Dataset 1 and Unstructured Dataset 2 and identifying variations between the two-dimensional structured representations of these datasets. Interactive visualization for these datasets can involve displaying both the structured tabular data and two-dimensional structured representations of the input datasets in the same space and in their native space.

While the above methods cite tabular data with numerical values, the above methods could apply to tabular data with arbitrary values including text.

The above description is illustrative and not restrictive. Many variations of the technology will become apparent to those of skill in the art upon review of this disclosure. The scope of the technology should, therefore, be determined not with reference to the above description, but instead should be determined with reference to the appended claims along with their full scope of equivalents.

1. A method for automated analysis of data obtained from biologic material, comprising:
extracting, from a visualization of the biologic material, first shapes that combine to form a target shape;
registering the first shape of the target shape to second shapes of a generic shape; and
identifying variations between the first shapes and the second shapes.

2. The method of claim 1, wherein the registering of the first shapes of the target shape to the second shapes of the generic shape comprises:
identifying marker points in the first shapes that correspond to generic marker points in the second shapes;
aligning the first shapes and the second shapes based on a first optimization function;
matching a first contrast of the first shapes with a second contrast of the second shapes by masking at least a portion of at least one of the first contrast and the second contrast; and
deforming at least one of the first shape and at least one of the second shape based on a second optimization function.

3. The method of claim 1, further comprising, prior to the extracting operation, processing input data associated with the visualization by:
rotating the visualization to a standard orientation;
homogenizing an intensity across the image; and
eliminating artifacts.

4. The method of claim 1, further comprising validating the registration by comparing an extracted feature from the visualization to a further extracted feature of a further visualization.

5. The method of claim 1, wherein the identifying operation comprises:
identifying local changes within the first shapes and the second shapes; and
evaluating the registering using a similarity metric.

6. The method of claim 1, further comprising:
displaying a further visualization of the target shape with data associated with the generic shape as a three dimensional representation;
wherein the data associated with the generic shape is displayed in the further visualization of the target shape in layers selectably displayable by a user;
wherein the data associated with the generic shape comprises name, function, and connection identifications; and
wherein the variations between the first shapes and the second shapes are displayed in the further visualization and are identified as abnormal based on a model.

7. The method of claim 1, wherein:
the first shapes comprise first graphlets, the first graphlets comprising first nodes and first segments; and
the second shapes comprise second graphlets, the second graphlets comprising second nodes and second segments.

8. The method of claim 1, wherein:
the first shapes comprise first volumetric objects; and
the second shapes comprise second volumetric objects.

9. The method of claim 1, wherein:
the generic shape is received from an atlas; and
the visualization of the target shape is obtained by one of Magnetic Resonance Imaging, Computerized Tomography scan, and a radiologic scan.

10. The method of claim 1, further comprising:
extracting, from a further visualization, third shapes of a further target shape to form a further target shape; and
registering the third shapes of the further target shape to at least one of the first shapes of the target shape and the second shapes of the generic shape.

11. A system for analyzing biologic material, comprising:
an extraction engine running on a processor coupled to a memory, the extraction engine extracting, in a visualization of the biologic material, first shapes that combine to form a target shape;
a registration engine running on the processor, the registration engine registering the first shape of the target shape to second shapes of a generic shape of a generic shape; and
an identification engine running on the processor, the identification engine identifying variations between the first shapes and the second shapes.

12. The system of claim 11, wherein the registering of the first shapes of the target shape to the second shapes of the generic shape comprises:
identifying marker points in the first shapes that correspond to generic marker points in the second shapes;
aligning the first shapes and the second shapes based on a first optimization function;
matching a first contrast of the first shapes with a second contrast of the second shapes by masking at least a portion of at least one of the first contrast and the second contrast; and
deforming at least one of the first shape and at least one of the second shape based on a second optimization function.

13. The system of claim 11, wherein data associated with the visualization is input to the extraction engine, the data being processed by the processor by at least one of:
rotating the visualization to a standard orientation;
homogenizing an intensity across the image; and
eliminating artifacts.

14. The system of claim 11, further comprising a validation engine adapted to validate the registration output by the registration engine by comparing an extracted feature from the visualization to a further extracted feature of a further visualization.

15. The system of claim 11, further comprising wherein the identification engine is adapted to:
identify local changes within the first shapes and the second shapes; and
evaluate the registering using a similarity metric.

16. The system of claim 11, further comprising a display adapted to display a further visualization of the target shape with data associated with the generic shape as a three dimensional representation, wherein:
the data associated with the generic shape is displayed in the further visualization of the target shape in layers selectably displayable by a user;
the data associated with the generic shape comprises name, function, and connection identifications; and
the variations between the first shapes and the second shapes are displayed in the further visualization and are identified as abnormal based on a model.

17. The system of claim 11, further comprising:
an atlas adapted to provide the generic shape; and
a database for storing the visualization of the target shape, the visualization being obtained by one of Magnetic Resonance Imaging, Computerized Tomography scan, and a radiologic scan.

18. A non-transitory computer-readable medium storing a program for analyzing biologic material, the program including instructions that, when executed by a processor, causes a processor to:
extract, in a visualization of the biologic material, first shapes that combine to form a target shape;
register the first shape of the target shape to second shapes of a generic shape; and
identify variations between the first shapes and the second shapes.

19. The non-transitory computer-readable medium of claim 18, wherein the program further includes instructions that, when executed, cause the processor to:
process input data, prior to the extract operation, associated with the visualization:
rotating the visualization to a standard orientation;
homogenizing an intensity across the image; and
eliminating artifacts;
validate the registration by comparing an extracted feature from the visualization to a further extracted feature of a further visualization;
wherein the registering of the first shapes of the target shape to the second shapes of the generic shape comprises:
identifying marker points in the first shapes that correspond to generic marker points in the second shapes;
aligning the first shapes and the second shapes based on a first optimization function;
matching a first contrast of the first shapes with a second contrast of the second shapes by masking at least a portion of at least one of the first contrast and the second contrast; and
deforming at least one of the first shape and at least one of the second shape based on a second optimization function; and
wherein the identifying of the variations comprises:
identifying local changes within the first shapes and the second shapes; and
evaluating the registering using a similarity metric.

20. The non-transitory computer-readable medium of claim 18, wherein the program further includes instructions that, when executed, cause the processor to:
display a further visualization of the target shape with data associated with the generic shape as a three dimensional representation;
wherein the data associated with the generic shape is displayed in the further visualization of the target shape in layers selectably displayable by a user;
wherein the data associated with the generic shape comprises name, function, and connection identifications; and
wherein the variations between the first shapes and the second shapes are displayed in the further visualization and are identified as abnormal based on a model.
